(19) United States
(12) Patent Application Publication
(10) Pub. No.: US 2013/0195925 A1
(43) Pub. Date: Aug. 1, 2013

(54) ANTI AGING APPLICATION AND METHOD FOR TREATING AGING

(71) Applicant: VASKIN, LLC, Jackson Heights, NY (US)

(72) Inventor: Khaleeq Y. ARSHED, Oyster Bay Cove, NY (US)

(73) Assignee: VASKIN, LLC, Jackson Heights, NY (US)

(21) Appl. No.: 13/650,940

(22) Filed: Oct. 12, 2012

Related U.S. Application Data
(60) Provisional application No. 61/555,289, filed on Nov. 3, 2011.

(51) Int. Cl. A61K 45/06

(52) U.S. Cl. CPC A61K 45/06
USPC 424/400; 424/62

(57) ABSTRACT

There is a topical anti-aging composition which is configured to interfere with the production of tyrosinase, as well as melanin by reacting with melanocytes as well as reacting with, or acting on keratinocytes. The anti-aging composition comprises an emulsion comprising a plurality of active ingredients comprising a plurality of pigmentation reducing elements which are configured to interrupt or prevent the production of tyrosinase and melanin. In addition there are a plurality of antioxidants a plurality of anti wrinkling agents a plurality of anti-inflammatory agents at least one additional agent comprising at least one of an emollient, a proactant and a conditioning agent.

### CROSS REFERENCE TO RELATED APPLICATIONS

This application is a non-provisional application that claims priority under 35 U.S.C. 119e from provisional application Ser. No. 61/555,289 filed on Nov. 3, 2011 the disclosure of which is hereby incorporated herein by reference.

### BACKGROUND OF THE INVENTION

### 1. Field of the Invention

One embodiment of the invention relates to a composition for inhibiting the aging of skin. As skin ages, it becomes thinner and more easily damaged. Intensifying this effect is the decreasing ability of skin to heal itself as a person ages. Among other things, skin aging is noted by a decrease in volume and elasticity. An example of the effects of aging are for example: laxity (sagging), rhytids (wrinkles), and the various categories of photo aging, including erythema (redness), dyspigmentation (brown discolorations), solar elastosis (yellowing), keratosis (abnormal growths), and poor texture.

The effects of aging can have an effect on multiple different layers of a person's skin.

There are five different layers to a person's skin: Stratum corneum; Stratum lucidum; Stratum granulosum; Stratum spinosum and Stratum germinativum/Stratum basale. Stratum corneum is the outermost layer while Stratum basale is the innermost layer. Keratinocytes are located in different layers of the skin. Melanocytes are located in the bottom layer or the stratum basale layer of a person's skin.

Discoloration or the altering of the skin color which is usually caused by photo aging is affected by the different pigments in a person's skin. There are at least five different pigments that determine the color of the skin. These pigments are present at different levels and places: 1) Melanin: It is brown in color and present in the germinative zone of the epidermis; 2) Melanoid: It resembles melanin but is present diffusely throughout the epidermis; 3) Keratin: This pigment is yellow to orange in color. It is present in the stratum corneum and fat cells of dermis and superficial fascia. 4) Hemoglobin: It is found in blood and is not a pigment of the skin but develops a purple color. 5) Oxyhemoglobin: It is also found in blood and is not a pigment of the skin. It develops a red color. Some compositions for inhibiting the aging of skin have been disclosed. For example, U.S. Patent Application Publication No. 2010/0271763 to Nakamura; U.S. Patent Application Publication No. 2007/0196296 to Osborne et al; U.S. Patent Application Publication No. 2004/0215006 to Bennett et al, US Patent Application publication no. 2009/0017141 to Rendon, U.S. Patent application Publication No. 2001/0158922 to Dupont et al, U.S. Patent Application Publication No. 2007/0196296 to Osborne et al, wherein the disclosure of these publications are hereby incorporated herein by reference in their entirety.

However, none of the above listed published applications disclose the features of at least one embodiment of the present invention which relies on the synergistic effect created by the interaction of a plurality of pigmentation reducing elements a plurality of anti-oxidants a plurality of anti-wrinkling agents and a plurality of anti-inflammatory agents acting on different stratum or layers of a person's skin to inhibit or reduce the effects of aging.

### SUMMARY OF THE INVENTION

In at least one embodiment of the invention there is a topical anti-aging composition which is configured to interfere with the production of tyrosinase, as well as melanin by reacting with melanocytes as well as reacting with, or acting on keratinocytes. The anti-aging composition comprises an emulsion or solution comprising a plurality of active ingredients comprising a plurality of pigmentation reducing elements which are configured to interrupt or prevent the production of tyrosinase and melanin. In addition, there are a plurality of anti-oxidants, a plurality of anti wrinkling agents, a plurality of anti-inflammatory agents, and at least one additional agent comprising at least one of an emollient, a proactant and a conditioning agent.

In at least one embodiment, the plurality of pigmentation reducing elements comprises at least one of: Tetrahydrocumin; Undecylenoyl Phenyl Alanine, Dimethylmethoxy Chromanyl Palmitate, Niacinamide, Kojic Acid Dipalmitate, Tetrahexydecyl Ascorbate, Glabridin, Mulberry bark Extract, Trans-Resveratrol.

In at least one embodiment, the plurality of anti-oxidants comprises: at least one of Co-Q10;Alpha Lipoic Acid; Aloe Vera Extract; Green Tea extract; DL-Alpha Tocopherol; Edelweiss Extract; or Tetrahydrocurcuminoid

In at least one embodiment, the plurality of anti-wrinkling agents comprises at least one of: Tripeptide-1, Hydrolyzed soy and Wheat Proteins; Date Palm Kernel Extract; Dimethylaminoethanol (DMAE); or Resveratrol/Trans-Resveratrol.

In at least one embodiment, the plurality of anti inflammatory agents comprises at least one of: Calendula; Chamomile; or white willow bark extract.

In at least one embodiment, at least one additional agent comprises at least one of an emollient, a proactant, and a conditioning agent comprising at least one of: Glycerine; allantoin; Sodium Lactate; Abyssinian Oil; Red Raspberry oil; water, Sorbitan Oleate, Glyceryl Stearate, PEG-100 Stearate, Cetearyl Glucoside, Stearyl Alcohol, Oleic Acid, & Glyceryl Monostearate.

In at least one additional embodiment there is also an oral supplement which is configured to be applied to the topical application as well. The oral supplement can comprise at least one of L-Glutathione and Vitamin C. In at least one embodiment, the oral supplement would include between 100-600 mg of L-Glutathione, (preferably 300 mg) and between 100-800 mg (preferably 400 mg) of Vitamin C encased in a liposome to provide for a time released delivery of the active ingredients.

In at least one embodiment, the active ingredients of the oral supplement reacts with the active ingredients of the topical application to inhibit the production of tyrosinase in melanocytes as well as reacting with the keratinocytes to prevent inflammation of a user's skin. The topical application works from the outside in on the skin while the oral application works from the inside out.

By using a topical application having the ingredients or composition as indicated above, this results in an application that can affect multiple different layers of a person's skin with different active ingredients acting on different layers of a person's skin to reduce the effects of aging.

By using both an oral and a topical application, active ingredients can be applied to a person's body which result in these active ingredients acting on multiple different layers of a person's skin in a synergistic effect to alter or inhibit the effects of aging.

Furthermore, in at least one embodiment, there is a process for inhibiting or reversing the signs of aging including at least one of the following steps: applying a cleanser, applying a toner; applying an anti-aging emulsion; ingesting an oral composition.

In addition, there is at least one additional embodiment that comprises a kit. The kit comprises at least one of a cleanser, a toner, an anti-aging topical composition, an oral composition, and an applicator for at least one of the cleanser, the toner, and the anti-aging topical composition.

By applying these different compositions at different times, the user can attack the aging of his or her skin from many different angles.

### BRIEF DESCRIPTION OF THE DRAWINGS

Other objects and features of the present invention will become apparent from the following detailed description considered in connection with the accompanying drawings which disclose one embodiment of the present invention. It should be understood, however, that the drawings are designed for the purpose of illustration only and not as a definition of the limits of the invention.

In the drawings wherein similar reference characters denote similar elements throughout the several views:

FIG. 1 is a flow chart of a process for administering a dermatological composition;

FIG. 2 is a schematic diagram of a cycle of the activation of pigment reducing agents;

FIG. 3 is a close up view of the first step in the process shown in FIG. 1 for the activation of the pigment reducing agents;

FIG. 4 is a close up view of the second step in the process shown in FIG. 1 for the activation of the pigment reducing agents;

FIG. 5 is a close up view of the third step in the process shown in FIG. 1 for the activation of the pigment reducing agents;

FIG. 6 is a close up view of the fourth step in the process shown in FIG. 1 for the activation of the pigment reducing agents; and

FIG. 7 is a close up view of the fifth step in the process shown in FIG. 1 for the activation of the pigment reducing agents;

FIG. 8 is a flow chart for applying the different components to enhance the anti aging properties of the skin; and

FIG. 9 is a view of a kit using the components of the invention.

### DETAILED DESCRIPTION OF THE PREFERRED EMBODIMENT

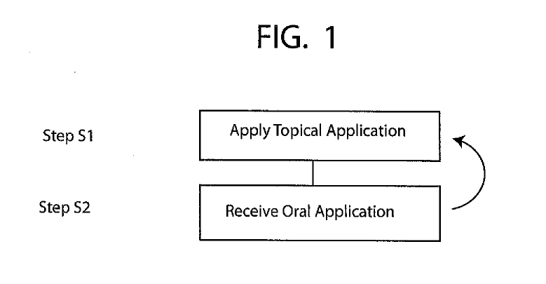

Referring in detail to the drawings, at least one embodiment of the present invention relates to an anti-aging system or dermatological system which can be in the form of a topical application comprising at least one of a cream, gel, oil, or similar type application, which be applied to the skin of a user. Alternatively, another embodiment involves a combination application which includes both a topical application in the form of a topical cream, gel, oil based application or similar type application which is used in conjunction with an oral supplement. This process is shown in greater detail in FIG. 1 which involves a first step S1 of administering the topical application, followed by the second step S2 of ingestion of a oral supplement. The use of the oral supplement would work in combination with the topical application. In at least one embodiment, the oral supplement would comprise at least one of L-Glutathione, Vitamin C, and Lipsome. For example, in at least one embodiment, the oral supplement would include between 100-600 mg of L-Glutathione, (preferably 300 mg) and between 100-800 mg (preferably 400 mg) of Vitamin C encased in a liposome to provide for a time released delivery of the active ingredients. Other embodiments could include other active ingredients as well and delivered in a capsule, or a capsule of only L-Glutathione, or only Vitamin C encased in a liposome.

In at least one embodiment, a person could take two capsules of the above ingredients twice a day after applying a topical application. In this way, a person could have the benefit of having both the active ingredients of the topical application work with the active ingredients of the oral application to produce an anti-aging effect on a person's skin.

Included herein is a description of the dermatological composition of the topical application and an outline of the benefits of this composition.

The compositions are configured to provide at least one composition which is configured to provide an anti-aging skin composition. This composition includes multiple different types of elements in at least the following fields: pigment reducing agents; anti-oxidants; anti wrinkle agents, firming and anti-aging agents; anti inflammatory agents; as well as non active ingredients used to aid in the application of these active ingredients, wherein these non-active ingredients comprise oils, water and emulsifiers.

### Pigment Reducing Agents

First, under the pigment reducing agents, there are the following components: Tetrahydrocumin; Undecylenoyl Phenyl Alanine, Dimethylmethoxy Chromanyl Palmitate, Niacinamide, Kojic Acid Dipalmitate, Tetrahexydecyl Ascorbate, Glabridin, Mulberry bark Extract, Trans-Resveratrol. Some of the benefits of each of these elements is provided below:

TetrahydroCurcumin (THC): Tetrahydrocurcumin has been shown to efficiently inhibit Tyrosinase. As such, it is an effective skin lightening agent with multifunctional topical benefits. It has been tested to determine its safety for topical use with no irritant or sensitization side effects. The anti-inflammatory effects of THC combined with their efficient antioxidant action render them useful as ingredients in anti-aging formulations, skin lightening and brightening products and topical formulations designed to maintain general skin health and integrity.

Undecylenoyl Phenyl Alanine: In vitro testing shows that Undecylenoyl phenylalanine works differently than other topical ingredients for spot reduction. Currently, Niacinamide and N-acetyl glucosamine work by interrupting the melanin synthesis pathway. Undecylenoyl phenylalanine works as an MSH (melanin stimulating hormone) antagonist, preventing the melanin synthesis from starting. In vitro testing shows that the combination of Undecylenoyl phenylalanine, N-acetyl glucosamine and Niacinamide show an additive effect in reducing melanin production without damaging skin cultures. It also suppresses the gene expression and protein release of stem cell factor in human keratinocyte cell culture and human, therefore reducing the pigment.

Dimethylmethoxy Chromanyl Palmitate: inhibits Tyrosinase & Melanogenesis without damaging the melanocytes.

Niacinamide: Niacinamide is the active form of Vitamin B3. A safe, nonirritating cosmeceutical ingredient which research shows can offer a number of benefits in improving the appearance of aged and photo damaged skin. Studies have shown topical Niacinamide to be effective in improving skin texture and the appearance of hyper pigmented spots, with improvement in overall blotchiness and the reduction of the severity of fine lines. It has been shown to stimulate collagen synthesis and production of ceramide, a lipid compound that contributes to the functional and structural integrity of the skin barrier. Theoretically, by improving barrier integrity, Niacinamide may help the skin become more resistant to external irritants. Nicotinamide prevents Immunosuppression caused by UVA and UVB radiation, and could be added to sunscreen.

Kojic Acid Dipalmitate: Some of the beneficial effects of Kojic Acid Dipalmitate involve skin lightening and color stability. Kojic Acid Dipalmitate offers more efficacious skin lightening effects. Compared with kojic acid, Kojic Acid Dipalmitate markedly enhances the inhibitory effects on tyrosinase activity, which prohibits the formation of melanin. Kojic Acid Dipalmitate is light and heat stable, while Kojic Acid tends to oxidize over time. Kojic Acid Dipalmitate is stable within a wide pH range of 4-9, which provides flexibility to formulators. Color Stability. Unlike kojic acid, Kojic Acid Dipalmitate does not turn brown or yellow over time for two reasons. However, kojic acid is not stable to light and heat, and tends to oxidize, which results in color change (often yellow or brown). Second, kojic acid tends to chelate with metal ions (e.g. iron), which often results in color change. On the contrary, Kojic Acid Dipalmitate is stable to pH, light, heat and oxidation, and does not complex with metal ions, which lead to color stability.

Tetrahexydecyl Ascorbate: Tetrahexydecyl Ascorbate is a very stable, oil-soluble Vitamin C ester which has anti-oxidant activity, inhibiting lipid peroxidation. Topical use can mitigate the damaging effects of UV exposure. Studies have shown it to stimulate collagen production as well as clarifying and brightening the skin by inhibiting Melanogenesis (the production of pigment) thereby promoting a more even skin tone. Unlike ascorbic acid, it will not exfoliate or irritate skin.

Glabridin has several properties that are useful for cosmetic applications. They include: Skin whitening property or the ability to inhibit Melanogenesis; Anti-inflammatory property; and Antioxidant properties. Glabridin is the main compound in the hydrophobic fraction of licorice extract and is known for its beneficial effects on the skin due to its anti-inflammatory and skin whitening properties. Glycyrrhizin and glychrrhetinic acid are also known to have anti-inflammatory properties. The hydrophobic fraction containing Glabridin and other flavonoids is known to have an inhibitory effect on Melanogenesis. Some researchers have established that this effect may be due to the constituents ability to inhibit tyrosinase activity. Both in-vitro and in-vivo studies were carried out to study the inhibitory effects of Glabridin on Melanogenesis and inflammation.

In a comprehensive study carried out by Yokota, T. et al., the inhibitory effects of Glabridin on Melanogenesis as well as inflammation were examined. The structure-function relationship of Glabridin was also studied. Topical skin-depigmentation activities of the active component, Glabridin, were examined using UVB-induced pigmented skins of brownish guinea pigs. A 0.5% Glabridin solution was applied topically to the skin. Topical application of Glabridin significantly reduced pigmentation induced by UVB radiation on the backs of the brownish guinea pigs. Skin samples were also taken from each of the Glabridin treated areas for histological studies. The treated tissue was stained with 0.1% DOPA and the inhibition of Melanogenesis was evaluated by counting the number of DOPA-positive melanocytes/mm2 under an optical microscope.

Research, including a study conducted by Tamotsu Takizawa, et al, published in “Toxicological Sciences” in June 2004, and another conducted by Hiroshi Suzuki, et al, and published in “Mutation Research” in June 2005, indicates that kojic acid may have some carcinogenic properties. Other studies on animals have shown brain, nervous system or behavioral side effects with high doses of kojic acid. A study by Mikio Nakagawa, et al, published in the journal “Contact Dermatitis” in January 1995, also confirmed that kojic acid can lead to dermatitis in some people. In addition, Kojic Acid may cause some increased skin sensitivity which may lead to a permanent or semi permanent case of dermatitis.

Therefore, to keep an effective skin whitening effect, additional skin whitening elements such as Glabridin have been added. This allows for the reduced amount of Kojic Acid Dipalmitate which thereby reduces the effects of the Kojic Acid Dipalmitate per application without thereby diluting the effects of whitening. For example, it is submitted that instead of using approximately 4 wt % per application of topical application, only 2 wt % is used because of the addition of Glabridin which also has the effect of whitening or at least depigmentation.

Mulberry Bark Extract: Mulberry fruit/bark extract is used cosmetically for its innovative skin-brightening and lightening properties. Used in conjunction with bearberry extract, licorice extract, wild berry extract and kojic acid, this has been proven to effectively lighten age spots and brighten the skin tone.

Resveratrol/Trans-Resveratrol: It reduces Melanogenesis Other ingredients provide additional anti pigmentation benefits. For example, these additional ingredients comprise any one of sunscreens, by blocking UV-A & UV-B, and therefore reducing Melanogenesis. Octocrylene, Benzophenone-3, and Red Raspberry Oil in the formulation provide this benefit.

In addition, at least one ingredient could be used for the suppression of the gene expression and protein release of stem cell factor in human keratinocyte cell culture and human, therefore reducing the pigment. An example of this type of ingredient is Undecylenoyl Phenyl Alanine. Furthermore, enhanced selective tyrosinase degradation targeted by fatty acids, which seem to act on the degradation of the enzyme during the physiologic proteasome-dependent mechanism, leading to the alteration of tyrosinase protein content in hyperactive melanocytes. Linoleic acid included in the formulation accelerates the process.

Furthermore, protease Activated Receptor 2 consists of G-protein coupled transmembrane receptors that are activated by serine proteases. PAR-2 is expressed in keratinocytes, not in melanocytes. Stimulation of this receptor enhances the phagocytosis rate of keratinocytes and leads to increased melanin transfer, which has been proved in vitro as well as in vivo. PAR-2 modulation affects pigmentation only when a keratinocyte-melanocyte contact is established. Ultraviolet irradiation induces PAR-2, and the other way round, blocking of the PAR-2 receptor inhibits UV-induced pigmentation. Inhibition of PAR-2 cleavage by serine protease inhibitors, completely avoids the UVB-induced pigmentation of epidermis analogs. Interference with the PAR-2 pathway has been shown to induce depigmentation by reducing the phagocytosis of melanosomes by keratinocytes, thus reducing melanin transfer. Natural soybeans contain Bowman-Birk inhibitor and soybean trypsin inhibitor, two serine protease inhibitors that interfere with the PAR-2 pathway, thereby reducing melanin transfer and inducing depigmentation. The effect can be obtained only with un-pasteurized soy milk. Furthermore, Aldinine included in the formulation interferes with PAR-2 pathway.

Another optional ingredient includes Niacinamide. Niacinamide is an effective skin lightening compound that down-regulates Melanogenesis by interfering with the interaction between keratinocytes and melanocytes. The influence of Niacinamide on melanosomes transfer was tested in a co-culture model of melanocytes and keratinocytes. The transfer of fluorochrome, which reflects the transfer of melanosomes, was confirmed visually by confocal microscopy and quantitatively by flow cytometry. The results showed that Niacinamide downregulated the amount of melanosomes transferred by approximately 35%-68%.

Skin turnover desquamation accelerates the loss of melanin by the peeling of stratum corneum cells. The capacity of these agents to disperse melanin pigment and/or accelerate epidermal turnover can result in skin lightening.

Therefore, chemical substances used as exfoliants, such as Alpha and Beta Hydroxy Acids, free fatty acids and Retinoic acid, and Citric Acid stimulate cell renewal facilitating the removal of melanised keratinocyte, leading to melanin pigment loss.

One of the goals of the formulation provided herein is to inhibit aging of a user's skin by reducing the natural production of melanin by a person in their body.

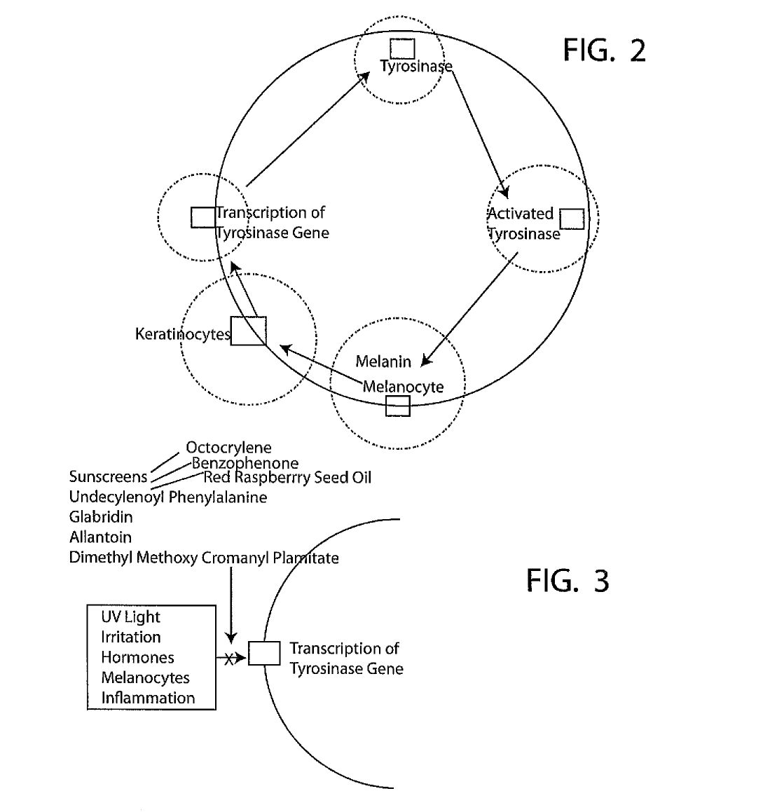

FIGS. 2-7 disclose the cycle of the Melanogenic cascade which results in the creation and synthesis of melanin. For example, FIG. 2 discloses the entire cycle of the melanogenic cycle which discloses first the transcription of the Tyrosinase gene which results from the receipt of UV light into a person's skin. This is shown in greater detail in FIG. 3 which shows that with the exposure to UV light, there are signals which cause irritation. Next, this activates hormones and results in melanocrytins, and MSH and ACTH. In addition this would normally result in inflammation of a person's skin. However, the cream provided, particularly the ingredients listed above as well as below serve to break this cycle. For example, sunscreens in the cream including octocrylene, benzophenone, and red raspberry seed oil serve to inhibit the growth or activation of these signals. In addition, the presence of undecylenoyl phenylalanine, Glabridin, allantoin, and dimethyl methoxy chromanyl palmitate results in further inhibition of the UV light signals activating, or acting on a person's skin.

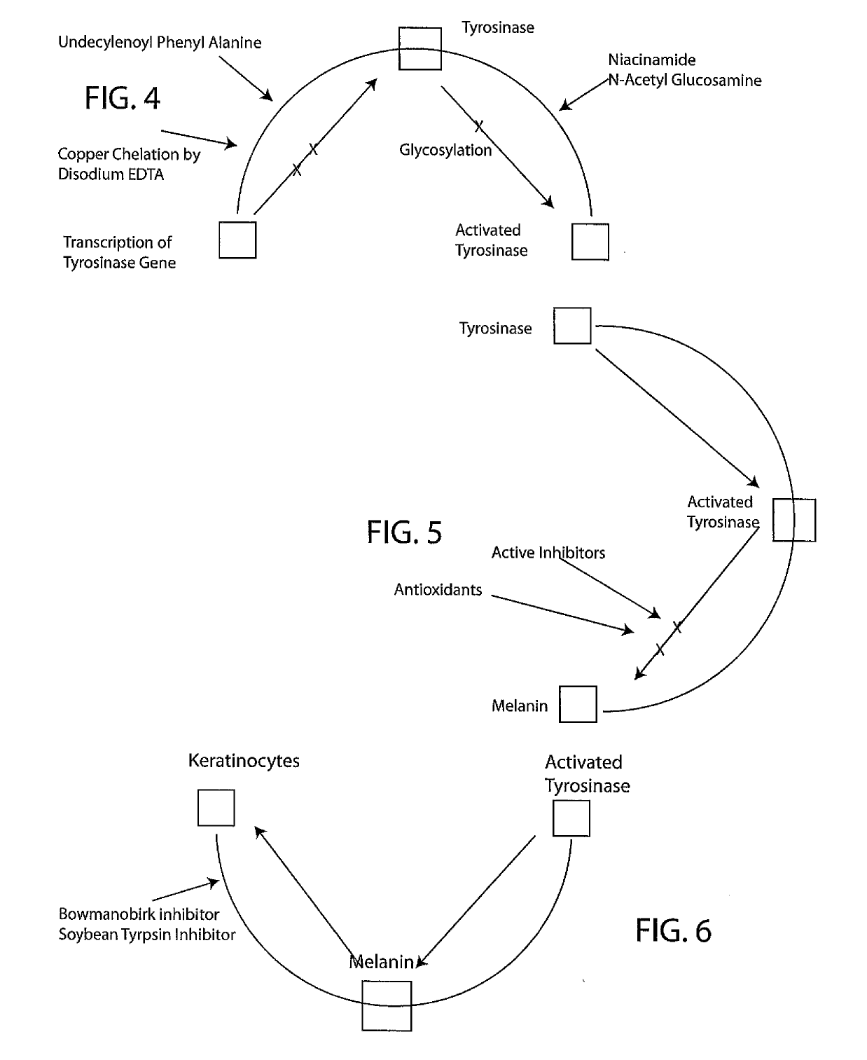

Next, to inhibit the production of tyrosinase, from the transcription of the tyrosinase gene, two components such as Undecylenoyl Phenyl Alanine, and EDTA can be used. In particular the copper chelation of disodium EDTA can be used to inhibit the production of tyrosinase. This is shown in greater detail in FIG. 4. This FIG. 4 also shows the interruption of glycosylation via the application of Niacinamide and N-Acetyl Glucosamine.

The interruption of the glycosylation then results in a reduced amount of activated tyrosinase. As shown in FIG. 5, the further interruption of the production of tyrosine from the activated tyrosinase is caused by the active inhibitors of Arbutin, Kojic Acid Dipalmitate, tetrahyhexyldecyl Ascorbate, mulberry extract, and glabradin. Additional interruption of this process is also effected by the presence of antioxidants such as CoQ10, alphalipoic acid, aloe Vera extract, green tea extract, di alpha tocopherol, edelweiss extract, tetrahydroxylcumenoid, and resveratrol.

FIG. 6 shows the interruption of the production of melanin. Melanin is packaged in melanosomes and is transferred to keratinocytes. To further inhibit the transfer of Melanin to Keratinocytes, Bowmen Birk inhibitors, can be used including soybean trypsin inhibitors including soy based serene protease inhibitors inhibit par 2 cleavage in keratinocytes causing phagocytosis of melanin keratinocytes.

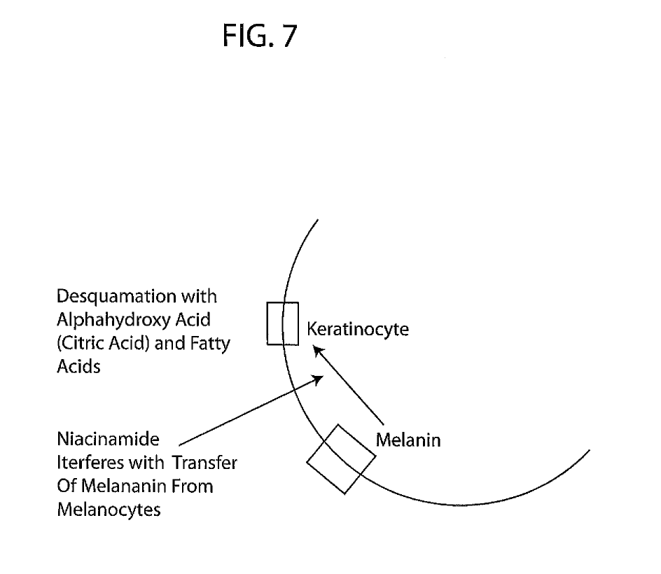

In addition as shown in FIG. 7, further inhibition of the melanin to the keratinocytes can be caused by desquamation. For example, this desquamation can be caused by alphahydroxy acid (citric acid) and fatty acids. Furthermore, Niacinamide interferes with the transfer of melanin from melanocytes to keratinocytes.

In addition, the entire cycle of melanagenic cascade is also affected at least partially by the components listed below as well.

Antioxidants

Antioxidants also play a significant role in reducing the pigment. Since the second reaction in the melanogenic cascade, i.e. the conversion of L3,4 dihydroxyphenylalanine to L3, 4 dihydroxyphenylalanine Quinone, as well as other nonenzymatic reactions in the pathway, are oxidative reactions, antioxidants such as MAP are also effective inhibitors of melanin synthesis in cultured melanoma cells. Compounds with redox properties can have depigmenting effects by interacting with quinones, thus avoiding the oxidative polymerization of melanin intermediates, or with the copper at the active site. Moreover, redox agents, by scavenging ROS (Reactive Oxidation Species) generated in the skin following UV (Ultraviolet) exposure, can inhibit possible second messengers which are able to stimulate epidermal Melanogenesis either directly or indirectly.

Because melanocytes are located in the stratum basale, and can directly effect the discoloration or pigmentation of the skin, the topical composition should be applied using a penetration enhancer. A Penetration enhancer like Tetrahydropiperine increases the efficacy of water soluble actives.

While antioxidants can be effective in reducing the pigmentation or discoloration of a persons' skin, these antioxidants can also be effective in reducing other aging effects on the skin. The aging of skin can also be due to the development of free radicals. Environmental factors cause the development of free radicals, which are known to accelerate aging. Most research has been done on ROS (Reactive Oxygen Species), a type of free radical. Yet another type, RCS (Reactive Carbonyl Species) have only recently been looked at, and the effect they have on skin deterioration and aging was also investigated. RCS are small molecular weight carbonyls and are especially dangerous, since they are byproducts of cellular metabolism including lipid peroxidation, glycation, autoxidation of sugars etc.

Free radicals cause DNA damage, proteasome degradation, as well as cellular and extracellular protein alterations and most importantly, they target and destroy collagen which we all need for firm young looking skin.

When collagen fibers are young, they are white, elastic and tough, yet with age, they lose their elasticity and crosslink and become yellow. It is the crosslinking of the collagen fibers that is responsible for the hardening of the collagen and the resultant skin aging and in this process RCS are directly responsible for the crosslinking of the collagen.

The skin can normally contend with HNE and neutralize it by means of the keratinocytes forming adducts with glutathione and in so doing neutralize the FINE.

However, when the skin has been subjected to sunlight, the glutathione is depleted and a person must look for another agent to neutralize the HNE and their RCS components.

The following is a list of the Antioxidant ingredient present in the formulation: CoQ10; Alpha Lipoic Acid; Aloe vera Extract; Green Tea extract; DLAlpha Tocopherol; Edelweiss Extract; Tetrahydrocurcuminoid.

CoQ10 (Coenzyme Q10), also known as ubiquinone, ubidecarenone, provides multiple benefits to the skin. CoQ10 is oil soluble, and fat soluble vitamin like substance and is therefore mobile in cellular membranes. The CoQ10 molecule holds electrons rather loosely, so this CoQ molecule will quite easily give up one or both electrons and, thus, act as an antioxidant. CoQ10 can have the effect of energizing the skin. Young skin has ample amounts of CoQ10 and so has lots of energy—energy needed to repair damage and make sure the skin cells are healthy. The antioxidant effects reduces the effect of photo aging. It rejuvenates skin by stimulating skin cell activity. Active skin cells get rid of toxins easily and can make better use of nutrients. When the skin ages, all these processes slow down causing dull and sallow, wrinkled skin. Boost your skin cell activity for younger looking skin with CoQ10. CoQ10 stimulates collagen production. Collagen is the protein that decreases as we age, leading to wrinkles and leathery skin. Helping you make more collagen, CoQ10 is a skin saving essential.

Another ingredient is Alpha Lipoic Acid. Alpha Lipoic Acid plays two very important roles in the cell. Firstly, it is a fundamental coenzyme in two vital reactions that lead to the production of energy at cellular level. These reactions are used with great success in skincare and cosmetic products. Secondly, it is a powerful antioxidant that is active against a broad spectrum of free radicals in both aqueous as well as lipid surroundings, and due to its size it can access nearly the entire body. Some of the functions of Alpha Lipoic Acid is that it is readily transported through cellular membranes and helps to recycle other antioxidants. For example, when vitamin E for instance quenches lipid peroxidation, and a vitamin E radical is formed, alpha lipoic acid will reduce it back to the active state of vitamin E. The same reaction occurs in the presence of the antioxidant, vitamin C. Alpha lipoic acid is active in both water and lipid environments, since the human cell consists of various parts, and these parts are either water or lipid based this particular ingredient can work in both to protect against free radical damage.

Free radical damage to the cell, and therefore the cells which form the skin, leads to premature ageing and skin wrinkling. If the concentration and presence of free radicals can be contained within the cell, far less oxidative stress and damage will be caused. This will result in aging being retarded at cellular level, as oxidative stress is one of the leading causes of aging.

Apart from fighting oxidative stress and reducing free radicals and their damage, alpha lipoic acid can also help boost the level of intracellular glutathione, which is another antioxidant that is very important to the immune system.

Applied topically to the skin, alpha lipoic acid has beneficial antioxidant properties and some research has been done in vitro on human dermal fibroblasts in cell culture systems.

Alpha lipoic acid is a powerful antioxidant that helps to increase the cell's metabolism and also inactivates hydroxyl and peroxyl radicals, singlet oxygen, hypochlorous acid, and nitric oxide, while at the same time acting as a catalyst and chelator. Alpha Lipoic Acid furthermore helps to fight inflammation, as well as edema (water retention), caused by such inflammation. It prevents the activation of NFkB (Nuclear Factor Kappa Beta), which in turn prevents proinflammatory chemicals called cytokines from being formed.

The activator protein 1 or transcription factor AP-1 is also activated in a healing manner by alpha lipoic acid. When AP-1 is activated by sunlight, it starts to produce collagen digesting enzymes called metalloproteinases, but when so activated by alpha lipoic acid, it actually destroys damaged collagen. Alpha Lipoic Acid also helps with the prevention of glycation where sugar and the protein of the collagen are cross-linked, leading to stiffness, wrinkles, lines and aging of the skin.

Another ingredient is Aloe Vera. The leaf juices of the aloe plant have important medicinal uses, making aloe one of the most respected medicinal plants found in many gels, creams and lotions. Modern researchers have identified several reasons why aloe gel spurs wound healing. It has antioxidant, antibacterial, antifungal and antiviral compounds that help prevent wound infections. It also has immune stimulating and anti-inflammatory compounds, and it stimulates collagen synthesis and skin regeneration. Aloe gel contains vitamins C and E, plus the mineral zinc. Aloe vera gel is soothing, anti-inflammatory, and antibacterial helps heal acnes and skin disorders of all kinds. Aloe brings cooling relief to fleabites, reducing itching and scratching, minor burns and rashes. Acemannan, a chemical compound found in Aloe Vera as a powerful immunostimulant.

Another ingredient is Green Tea Extract. Green tea has particular cosmetic aiding properties. For example, the tannins of green tea interact with proteins and are astringent and also have antioxidant activity. One of the polyphenols in green tea Epigallocatechin galate (EGCG) is thought to be 200 times more powerful than vitamin E for neutralizing free radicals.

Since free radicals can attack and damage the DNA of the cells, the antioxidant effect of green tea has a protective role to play as well, since it prevents free radical damage and possible skin tumors and cancers and can be used in confidence as an anti-aging ingredient in skin care preparations.

It has also been showed that the EGCG in green tea is effectively absorbed via the skin. Green tea is a potent anti inflammatory agent thereby reducing inflammation in the skin, and most importantly having an inhibitory action on collagenase, which is a collagen reducing enzyme that breaks down collagen.

Since green tea helps to inhibit collagenase, the topical use of green tea helps to promote the health and quantity of collagen, thereby maintaining a firm and elastic skin.

In one clinical trial it was shown that the skin had measurably more elastic tissue content after continuous application of a product containing green tea.

The flavonoids and catechols in green tea provide it with vitamin P properties and the tannins in its chemical composition give it astringent properties, whereas the polyphenolic compounds also act as an astringent, but also protect the skin.

The Methylxanthines contained in green tea also stimulates skin microcirculation and therefore positively influence the tone and health of the skin. Green tea also exhibits a photo protective effect when applied to the skin and reduces erythema formation.

In some studies it also showed that polyphenols found in green tea limited cell death when exposed to radiation, thereby showing that these polyphenols have a cell protecting function as well.

Another ingredient comprises Vitamin E in the form of DLAlpha Tocopherol. Tocopherol has very specific benefits to the skin. For example, it protects the skin from environmental pollution, it has a protecting action against UV radiation—although it cannot be classed as a sunscreen. It is an excellent moisturizer It contains powerful anti-inflammatory action—and this prevents premature aging, as inflammatory conditions in the skin is a leading cause of skin aging. It also has excellent wound healing properties. Apart from all of these excellent qualities, vitamin E also enhances and helps with the penetration of other compounds into the skin and is itself well absorbed by human skin.

Vitamin E has a natural affinity to the skin but the amount of vitamin E is reduced in the skin when exposed to sunlight.

Environmental pollutants, as well as oxidative material in the body, cause the formation of “free radicals” which are unpaired oxygen molecules, which wreaks havoc on the cells, and cause extensive damage, which directly results in premature aging.

Vitamin E is a very effective antioxidant, chemically mediated by the phenolic OH group of the chromane ring, and helps to remove the formed “free radicals” and thereby ensures that oxidative damage does not occur, by disrupting the chain reaction caused by the free radicals.

Vitamin E can also be very effective in controlling the harmful effects of UV radiation on a person's skin. UV radiation from the sun may be great for getting a tan, and for the body to make its own vitamin D, yet exposure to the rays also cause an array of negative reactions in the skin. The first and most well known in the short term is sunburn (erythema) which leads to degenerative changes within the cell, causing age spots and leading to the loss of elasticity, which in turn will result in dry coarser skin, as well as wrinkles and lines.

Another factor that must be considered with UV radiation from the sun is that it causes the skin to become photosensitive and also cause cancerous growth—starting with actinic keratosis or solar keratosis and then developing further to basal cell and squamous cell cancer, as well as malignant melanoma.

When vitamin E is applied to the skin, a reduction of erythema, sunburn cells, chronic UVB-induced skin damage, and photo carcinogenesis is experienced

Vitamin E has also proven effective in reducing the amount of skin tumor formation along with having the properties of prevention of sun damage.

While there is no absolute protection from the sun, cosmetics and moisturizers containing antioxidants, such as Vitamin E, Vitamin C and Beta Carotene, can counteract damage to cell membranes, DNA, and skin proteins due to oxidation caused by sunlight.

These materials don't work like sunscreens, but they help minimize the damaging reactions in the skin, especially when used in conjunction with sunscreens. When vitamin E is combined with vitamin C, and applied to the skin, it helps to reduce the formation of sunburn cells and offers good photo protection.

Other studies have also shown that vitamin E protects the fibroblast in the skin—these are the cells that make collagen, glycosaminoglycans, as well as the elastic fibers and glycoproteins found in the extracellular matrix of the skin.

It is also important to remember that when exposing the skin to the sun—the resultant burn or tan is an inflammatory reaction of the skin, and in the quest for achieving a youthful looking skin, it is of prime importance to prevent and to reduce any inflammatory conditions.

The topical application of vitamin E has also proven to be effective in reducing sun induced skin wrinkling and has excellent skin moisturizing properties, also helping to prevent transepidermal water loss by the skin, thereby increasing the natural moisture content of the skin.

Vitamin E has also proven effective at preventing premature skin aging, excessive crosslinking and DNA degeneration.

Another factor that plays a major part in skin aging is crosslinking of protein that occurs in the skin—which is even more prevalent when the skin is subjected to UV radiation.

In this case, vitamin E has also proven to be effective in preventing excessive crosslinking and resultant aging, as well as protecting the natural enzymes in the skin

As we age, the degeneration of DNA—which carries the blueprint of our cells, can occur, and such damage to the DNA can also result not only in skin problems, but also in premature aging. It has been shown that topical application of vitamin E also prevents the degeneration of DNA, especially when compromised by photo damage.

Vitamin E acts as a fat (lipid) based antioxidants. Vitamin E protects the cells and cell membranes from this oxidative stress. This oxidative stress is also caused by normal cell metabolism, but is exasperated by a variety of environmental factors, including UV radiation, pollution, tanning, smoke, heavy metals in the environment etc.

Vitamin E is normally found in the skin, but exposure to sunlight has been shown to deplete this extremely important antioxidant and topical application of it boosts the availability of it. To have adequate tocopherol is even more important in mature skin, as the levels of it seems to decrease with age, when it is needed even more to fight the ravages of free radicals and in so doing, help in the battle against ageing. Apart from the antioxidant properties, it also helps to moisturize the skin and even more importantly helps with tissue repair, thereby keeping the skin in good condition.

Vitamin E is also quite beneficial in the field of wound healing. For example, in tests done, it was found that a solution containing 5% of Vitamin E acetate decreased the healing time required, and also that vitamin E increases the breaking strength of wounds. Vitamin E is required for healthy collagen in the skin—which is the support system in the skin and helps the skin to remain firm and healthy.

Tocopheryl acetate is the ester of Tocopherol (q.v.) and acetic acid and is classified as both an ester and heterocyclic compound. It is used as an antioxidant and a miscellaneous skin conditioning agent and is known by its more common name, vitamin E although international cosmetic labeling regulations prohibit the word “vitamin” on the label of cosmetic products.

Another optional ingredient is edelweiss extract.

Some of the active ingredients of edelweiss extract are bisabolane, sitosterol, tannin, chlorogenic acid, apigenin7glucoside, luteolin, luteolin4glucoside. These components are preserved with vegetal glycerin, butylenglycol, ethanol. Very high radical scavenging activity of antioxidant products of 286 (=Radical Protection Factor, RPF). Antioxidant activity is twice as much as vitamin C.

It is a potent antioxidant with very effective anti-aging, anti-inflammatory & antiseptic properties, that protects the skin before & after sun exposure. Together with sun filters, skin protection is enhanced further.

Another ingredient can include Tetrahydrocucumenoids. Tetrahydrocurcuminoids are phenolic in nature, acting as antioxidants, protecting against free radicals and preventing the generation of free radicals. Unlike yellow Curcuminoids, THC is a colorless compound which can therefore be used in color free cosmetic products that currently employ conventional and synthetic antioxidants.

Curcuminoids offer protection to the skin and should be considered for inclusion as functional antioxidants in topical preparations. THC scavenges free radicals that are generated through exposure to ultraviolet radiation, chemicals or other environmental stress factors which age the skin. The efficacy of Curcuminoids and THC is also concentration dependant, with THC being even more relatively effective at lower concentrations. THC also helps to improve the shelf life of topical formulations by inhibiting the auto oxidation of fats by offering protection against rancidity of the fat components used in the formulation of cosmetics.

Tetrahydrocurcumin has been shown to efficiently inhibit Tyrosinase. As such, it is an effective skin lightening agent with multifunctional topical benefits. It has been tested to determine its safety for topical use with no irritant or sensitization side effects. The anti-inflammatory effects of THC combined with their efficient antioxidant action render them useful as ingredients in anti-aging formulations, skin lightening and brightening products and topical formulations designed to maintain general skin health and integrity.

### ANTIWRINKLES, FIRMING & ANTI-AGING AGENTS:

The following components which relate to anti wrinkling can also have an effect on the other characteristics of aging as well.

1: Tripeptidel, Hydrolyzed soy and Wheat Proteins. Although Reactive Oxygen Species (ROS) have been known and studied for years, Reactive Carbonyl Species (RCS), another important class of intermediates, have only recently been related to skin deterioration. RCS are small molecular weight carbonyls that are activated by a, B insaturation (such as 4hydroxynonenal and acrolein), aoxo substitution (such as glyoxal) and Boxo substitution (such as malondialdehyde).

RCS are especially dangerous because they are a byproduct of cellular metabolism, including lipid peroxidation, glycation, auto oxidation of sugars, etc. Among the damage caused by RCS we find DNA damage, proteasome degradation as well as cellular and extracellular protein alteration. This last effect has been linked to skin deterioration, and indeed skin collagen, the primary component of the extracellular matrix, is an important target for RCS.

As stated above, young collagen fibers are tough, elastic, and in bulk appear white, but with age they undergo crosslinking, lose their elasticity and become yellow. Crosslinking is responsible for the hardening of collagen as well as for the deterioration of skin with age, and the molecules responsible for crosslinking are RCS.

RCS, especially dicarbonyl compounds, react with proteins to form a variety of adducts in a reaction known as the Maillard reaction. These adducts are collectively known as Advanced Glycation End products (AGEs) and are effective indicators of protein damage and are highly increased with age.

If we concentrate on HNE (hydroxynonenal), one of the most abundant and toxic of these aldehydes, we see that HNE protein adducts are detected in photo damaged and aged skin but not in young healthy skin. Keratinocytes detoxify endogenously formed HNE by forming adducts with GSH (glutathione), the skin's natural hydrophilic antioxidant.

However, when submitted to UVB, keratinocytes are depleted of GSH (Glutathione), they can no longer detoxify from HNE and they die. So an external agent is needed to quench HNE and other RCS when keratinocytes receive UVB, that is, when the skin is exposed to the sunlight.

GHK is able to covalently sequester FINE and other reactive aldehydes, thereby acting as a cellular detoxifier. The tripeptide is more active than carnosine, a dipeptide (Balanyl L histidine) present in high concentration in skeletal muscle, which also acts as a cellular scavenger.

The hydrolyzed vegetable protein (HVP) is a stimulator of Collagen III as proved by in vitro tests on fibroblasts. Collagen III is the type of collagen produced by extremely youthful skin. When you are 50 years old, approximately 90% of your collagen is Collagen I, when you were 4 years old, approximately 90% of your collagen was Collagen III. During the aging process, cells gradually lose their ability to produce Collagen III through functional impairment.

The key attributes of HVP include: Anti-Wrinkle; Targets Expression Wrinkles; Enhanced Skin Tone; Skin Firming and Smoothing; Collagen Synthesis; Promoting Extra Cellular Matrix; Anti-inflammatory. The HVP boosts Collagen III production by almost 300% in 7 days, while it does not affect Collagen I, already plentiful in the skin.

Date Palm Kernel Extract: Hormones have an important role in skin appearance and the levels of hormones have a part in skin aging and wrinkling. There has been increased interest recently in the use of natural substances such as phytohormones in anti wrinkle products like date palm kernel extract. Topical date palm kernel at a concentration of 5 percent reduced the area covered by wrinkles by 27% and reduced the depth of remaining wrinkles by 3.5%. Upon visual inspection 60% of the volunteers seemed significantly improved in appearance. This shows date palm kernel has anti-wrinkle effects in skin care products.

Dimethylaminoethanol (DMAE) DMAE is an analog of the B vitamin choline and is a precursor of acetylcholine. Although the role of acetylcholine as a neurotransmitter is well known, growing evidence points to acetylcholine as a ubiquitous cytokine like molecule that regulates basic cellular processes such as proliferation, differentiation, locomotion, and secretion in a paracrine and autocrine fashion. Indeed, this modulatory role may contribute to the cutaneous activity of DMAE. In a randomized clinical study, 3% DMAE facial gel applied daily for 16 weeks has been shown to be safe and efficacious in the mitigation of forehead lines and periorbital fine wrinkles, and in improving lip shape and fullness and the overall appearance of aging skin. Conclusions: Thus, the benefits of DMAE in dermatology include a potential anti-inflammatory effect and a documented increase in skin firmness with possible improvement in underlying facial muscle tone.

DMAE (dimethylaminoethanol), is a naturally occurring substance that facilitates the synthesis of a neurotransmitter acetylcholine. DMAE also may stimulate the synthesis of phosphatidylcholine, an important component of cell membranes. Interestingly, DMAE is far better known and researched as a “smart drug” than a skin firming agent. In a number of studies, DMAE has been shown to reduce age related decline in cognitive ability and memory. It was also found to modestly increase life span of laboratory animals. An intriguing finding in some DMAE studies was that it reduced the accumulation of lipofuscin deposits inside cells. Lipofuscin is a cellular pigment consisting of aggregated chunks of molecular waste. It tends to occur in the cells of older people. It is likely that lipofuscin is not simply a byproduct of aging but also contributes to the aging process. Neurons, heart and skin of older people usually contain particularly large amounts of lipofuscin.

DMAE can cause some degree of skin tightening. The effect of DMAE is often noticeable although seldom dramatic. Some people report a cumulative effect with continued use of DMAE.

Resveratrol/TransResveratrol: Remarkable advances in anti-aging applications for skin resilience, including reductions in fine lines and improvement in skin tone, are being seen with the addition of Metabiotics™ Resveratrol to cosmetic formulations. Produced through fermentation of the small molecule, resveratrol, with the microorganism, Pichia pastoris, this innovative biotechnological ingredient is designed for use in anti-aging skin creams and related products.

Pichia pastoris, the microorganism used in the production of Metabiotics™ Resveratrol, was chosen chiefly due to its long history of use in the pharmaceutical industry where it is considered GRAS or generally recognized as safe by the FDA. Only recently has it been explored for its use in personal care applications. Pichia pastoris, like others of the Saccharomycetase genus to which it belongs, has the distinctive ability to express bioactives that are capable of unique benefits, without application of human cell lines. Uniquely, it also has an affinity for lipid based compounds, therefore acting as a model organism for biofermentation of oil soluble or insoluble actives like resveratrol, which has presented formulating challenges due to its water solubility and stability limitations.

Collagen IV and anti-inflammatory synthesis activity is afforded through the complete metabolization of the resveratrol in the biofermentation process, targeting microinflammation and maintaining the structural integrity of skin cells through the dermal epidermal junction (DEJ). An ideal active to incorporate in cosmetic formulations designed to ameliorate the visible signs of aging, Metabiotics™ Resveratrol promotes firming and helps rebuild DEJ structural integrity.

In vitro studies were performed to determine the inhibitory benefits of Metabiotics™ Resveratrol on a full thickness tissue model to reduce expression of the anti-inflammatory enzyme cyclooxygenase 1 [COX1] along with its ability to promote the synthesis of Collagen IVA on the same tissue model. The results, in both cases, confirm with statistical significance that either a 1% or 2% treatment of Metabiotics™ Resveratrol reduces the levels of the COX1 enzyme and increases the amount of collagen WA synthesis. In vivo, an anti-aging study has confirmed a reduction in fine lines and wrinkles following a 28 day application, as well as firming and lifting of the skin to reduce notable sagging that was present prior to treatment.

In addition, other anti inflammatory agents can be used. This can include Calendula oil. Calendula oil is classed as macerated or infused oil, where the plant material is infused in oil. The botanical material is later removed, leaving the oil with some of the properties of the botanical material. Calendula is used in skincare, for its soothing properties and to assist with circulation.

It is the combination of the lipophilic (oil) extracts and the hydrophilic extracts which contains flavonoids and saponins that has shown to promote healing and skin repair, while having excellent anti-inflammatory properties.

Apart from the great anti-inflammatory properties, it also has good vulnerary properties (preventing tissue degeneration while arresting bleeding in wounds), making it excellent to help with stubborn wounds, ulcers, bedsores, varicose veins, bruises, rashes, eczema etc. The ancient Greek and Roman civilizations used Calendula as a medicinal herb, but it should not be confused with Tagetes oil, which is an essential oil made from Calendula erecta (African or Aztec marigold, cempazuchil).

Calendula oil is great to help with sore, inflamed and itchy skin conditions, for burns, eczema and nappy rash, as well as sore cracked nipples. It is also used to treat thread and spider veins as well as varicose veins.

The properties of calendula are anti-inflammatory, styptic, antiseptic, anti hemorrhagic. It is used in the treatment of ulcers and inflamed cutaneous lesions as well as slow healing wounds and bruising. The main constituents are volatile oil, saponins, flavonoids, clendulin, sterols, fatty acids, calendic acid, oleanic acid, triterpenoids, tocopherols and flavonol glycosides. Apart from the anti-inflammatory effect that it has, it is also most effective in healing sores and wounds and in fighting fungal infections such as Athletes foot. It has been shown that Calendula stimulates the regeneration of wound tissue, since it intensifies the metabolism of glycoproteins, nucleoproteins and collagen during the healing process.

Chamomile contains phenolic derivatives, which have an antiseptic and healing quality, which is most helpful in relieving skin congestion. These soften the skin, making it an effective ingredient in any anti-aging cosmetic product. Over the past 30 years, extensive scientific investigations have confirmed the traditional uses of German chamomile (Matricaria recutita), making it one of the most popular and versatile essential oils in the world. It is one of the most widely used botanicals and has carminative, emollient, healing, tonic, antioxidant and anti-inflammatory properties. It contains azulene which helps to reduce puffiness and cleanse pores of impurities.

The alphabisabolol in the oil promotes granulation and tissue regeneration. Chamomile oil has also great wound healing properties and is extensively used with skin problems such as rashes, acne, eczema, psoriasis, hypersensitive skin and allergic conditions.

The flavonoids as well as the lipophilic (sesquiterpenes) compounds found in camomile oil help to reduce capillary fragility, which in turn helps to strengthen the skin. The vasoconstrictor properties again help to reduce redness of the skin, which is due to enlarged capillaries. Compounds found in chamomile contain more than 100 constituents and typically includes chamazulene, alphabisabolol, bisabolol oxide A, bisabolol oxide B and bisabolene oxide A. Other compounds such as apigens, borneol, farnesol, furfural, matricarin, as well as chlorogenic acid are present, but the main chemical components important to skincare are the farnesene, cuminic and azulene found in the oil

Classification of chamomile as a cosmetic ingredient include Chamomilla recutita (Matricaria) flower oil which is the volatile oil distilled from the dried flower heads of Matricaria recutita. It is classed as an essential oil and is used as a miscellaneous skin conditioning agent and fragrance ingredient. It is also known as German chamomile oil, Hungarian chamomile oil, Matricaria flower oil, oil of Matricaria, blue chamomile, as well as wild chamomile oil.

White Willow Bark Extract. Willow bark extract (Salix alba) is used in skincare as an astringent and has antiseptic, anti-inflammatory and antipyretic properties. It is also used to stimulate circulation. The main constituents of willow bark extract are phenolic acids; such as salicin, salicortin, fragilin, populin, triandrin and vimalin, as well as flavonoids, tannins (gallotannins and catechintype tannins) and minerals.

It also contains salicylic acid, which stimulates new cell formation by helping the old skin to slough off Willow bark can be classified as a cosmetic ingredient wherein it is an extract of the bark of the white willow, Salix alba and is classed as a biological product. It is used as an occlusive skin conditioning agent.

### Emoliants, Proactants, and Conditioning Agents.

To aid in the application of the first mixture or composition which is applied topically, such as a cleanser or cream the composition can include emoliants, Proactants, and conditioning agents. One of these emollients can include propylene alcohol which can be naturally derived from vegetable oils. Other elements can include glycerin which are replaced more and more by its cheaper counterpart, sorbitol. Glycerin helps to maintain the water balance in the intercellular matrix and in so doing it maintains the skin's homeostasis. Glycerin is a polyhydric alcohol and is classed as a polyol. It is used as a fragrance ingredient, humectant, skin conditioning agent, skin protectant and viscosity decreasing agent. It is also known as glycerine, glycerol, glycyl alcohol, as well as 1,2,3Propanetriol and 1,2,3Trihydroxypropane.

Another ingredient can be allantoin. Allantoin is a synthetic free flowing Hygroscopic powder (naturally occurring in comfrey) widely used in cosmetic, dermatological and pharmaceutical formulations for its soothing and anti-irritating properties. Allantoin stimulates healthy, normal tissue formation even at low concentrations. Allantoin has been classified by the FDA as an Over The Counter (OTC) Category I (safe and effective) active ingredient skin protectant. It is FDA approved for this use at 0.5% to 2.0% in formulations. Allantoin is nontoxic, nonirritating, and nonallergenic.

Another ingredient can be include sodium lactate. In cosmetic applications, it exhibits strong anti bacterial & humectants properties.

Another ingredient can be Abyssinian oil. Abyssinian oil is natural seed oil with an ultra light, nongreasy skin feel with excellent emollient properties. It contains a high percentage of unsaturated C22 fatty acids, making it highly resistant to oxidation. Liquid at room temperature even though of a high molecular weight, it is very stable against heat breakdown. As a lipid seed oil, it is easily incorporated in any water in oil or oil in water formula via the oil phase. It offers easy spreadability with no tack. It is non-GMO vegetable plant derived oil with a mild, pleasant odor. It is biodegradable and not toxic and has not been tested on animals.

Another ingredient can be red raspberry oil or cold pressed virgin oil that contains a high 83% of Essential Fatty Acids or EFA's, the most abundant of which are linoleic, alpha linoleic, and oleic acids. The composition of these omega3 and omega6 fatty acids has been shown to reduce the effects of oxidative stress in skin. Red raspberry seed oil can be used as a free radical scavenger and antioxidant. Red raspberry seed oil is a rich source of alpha and gamma tocopherol as well as ytocopherol, along with carotenoides (Vitamin A). With a reported use for the prevention of rash, eczema and other skin lesions, it also has superior anti-inflammatory properties as compared to other well-known oils such as virgin avocado oil, grape seed oil, hazelnut oil, and wheat germ oil (Oomah, et al 2000).

Notably, the red raspberry seed oil may act as a broad spectrum UVA and UVB shield. Red raspberry seed oil can have a protection factor equal to titanium dioxide and a potential SPF between 28-50, it thus aids in sun screen formulations.

Other emulsifier ingredients include Sorbitan Oleate, Glyceryl Stearate, PEG100 Stearate, Cetearyl Glucoside, Stearyl Alcohol, Oleic Acid, & Glyceryl Monostearate.

The following are examples of components that can be used in a topical application such as with a cream or cleanser. The cream or cleanser can be used with other topical applications such as with a toner and/or astringent, or cleanser, and also be used with oral applications such as a pill, capsule or liquid as well.

### EXAMPLES

While the composition can be provided in a particular, range a first example or at least a preferred embodiment is provided below wherein this example includes a topical application composition such as for a cream. The compositions are listed below in wt %:

### Example 1

| | |
|---|---|
| Deionized Water | 44.00 |
| Allantoin | 0.50 |
| Na2 EDTA | 0.20 |
| Xanthan Gum | 0.50 |
| Sodium Lactate | 1.50 |
| Citric Acid | 1.00 |
| Glycerine 99.5% | 2.50 |
| Tetrahydrocurcuminoid | 0.25 |
| Tetrahydropiperine | 0.25 |
| Glabridin | 0.50 |

Oil Phase

| | |
|---|---|
| Sorbitan Oleate | 3.50 |
| Glyceryl Stearate/PEG100 Stearate | 3.50 |
| Cetearyl Glucoside | 2.00 |
| Stearyl Alcohol | 0.50 |
| Oleic Acid | 0.50 |
| Glyceryl Monostearate | 2.00 |
| Red Raspberry Seed oil | 2.00 |
| BHT | 0.05 |
| Dimethicone 200/350 | 1.5 |
| Abyssinian Oil | 1.5 |
| CoEnzyme Q10 | 2.50 |
| Dimethylmethoxy Chromanyl Palmitate | 1.00 |
| Undecylenoyl Phenyl Alanine | 1.00 |
| Tetrahexydecyl Ascorbate | 2.00 |
| Finsolv TN | 1.70 |
| Octocrylene | 5.00 |
| Benzophenone3 | 1.5 |

Emulsion Phase

| Niacinamide | 1.00 |
|---|---|
| Water | 6.00 |
| Kojic Acid Dipalmitate | 0.50 |
| Ingredients: | Actual % |
| Aloe Vera Powder | 0.50 |
| Date palm kernel Extract | 1.50 |
| Mulberry Bark Extract | 0.20 |
| Edelweiss Extract | 0.20 |
| Alpha Lipoic Acid | 1.00 |
| DMAE | 1.00 |
| Sunspheres SPF Booster | N/A |

Low Temp Extracts

| | |
|---|---|
| DLAlpha Tocopherol | 1.00 |
| Calendula Flower Extract | 0.20 |
| Chamomile Flower Extract | 0.20 |
| White Willow Bark Extract | 0.20 |
| Red Clover Flower Extract | 0.20 |
| Resveratrol | 1.00 |
| Elder Flower Extract | 0.20 |
| Aldenine (0.1% GHK Tripeptide1) | 1.00 |
| (Hydrolyzed Soy &Wheat Protein) | |

Preservatives/Fragrance

| | |
|---|---|
| PhenoxyEthanol | 1.00 |
| DMDM Hydantoin | 0.15 |
| Fragrance | N/A |

The composition of the above topical example can be used to form a first mixture which can then be applied with a second mixture administered orally. This second mixture can comprise L-Glutathione and/or Vitamin C. L-Glutathione can be present in the range of 100-600 Mg, while Vitamin C can be provided in a range of 100-800 mg. In at least one embodiment the second mixture can comprise 300 mg of L-Glutathione and 400 mg of Vitamin C.

Some of the non-essential components of the cream and which can be easily taken out can include:
Dimethicon
Finsolve TN
Kojic Acid
Calendula flower Extract
Elder Flower Extract

### Example 2

### Another Example of the Topically Applied Mixture is Provided, Such as for a Cream in a Range of Wt % Composition

| | |
|---|---|
| Deionized Water | 3555 |
| Allantoin | 0.251.5 |
| Na2 EDTA | 0.20.4 |
| Xanthan Gum | 0.251.0 |
| Sodium Lactate | 0.502.0 |
| Citric Acid | 0.53.0 |
| Glycerine 99.5% | 0.254.0 |
| Tetrahydrocurcuminoid | 0.11.0 |
| Tetrahydropiperine | 0.11.0 |
| Glabridin | 0.251.0 |
| Sorbitan Oleate | 2.05.0 |
| Glyceryl Stearate/PEG100 Stearate | 1.05.0 |
| Cetearyl Glucoside | 1.05.0 |
| Stearyl Alcohol | 0.253.0 |
| Oleic Acid | 0.252.5 |
| Glyceryl MonoStearate | 0.55.0 |
| Red Raspberry Seed oil | 0.55.0 |
| BHT | 0.012.0 |
| Dimethicone 200/350 | 0.54.0 |
| Abyssinian Oil | 0.255.0 |
| CoEnzyme Q10 | 0.254.0 |
| Dimthylmethoxy Chromanyl Palmitate | 0.253.0 |
| Undecylenoyl Phenyl Alanine | 0.252.0 |
| Tetrahexyldecyl Ascorbate | 0.503.0 |
| Finsolv TN | 0.53.0 |
| Octocrylene | 3.07.0 |
| Benzophenone3 | 0.52.5 |
| Niacinamide | 0.52.0 |
| Kojic Acid Dipalmitate | 0.252.0 |
| Aloe Vera Powder | 0.252.5 |
| Date palm kernel Extract | 0.53.0 |
| Mulberry Bark Extract | 0.130 |
| Edelweiss Extract | 0.12.0 |
| Alpha Lipoic Acid | 0.53.0 |
| DMAE | 0.53.00 |
| Sunspheres SPF Booster | |

Low Temp Extracts

| | |
|---|---|
| DLAlpha Tocopherol | 0.53.0 |
| Calendula Flower Extract | 0.11.5 |
| Chamomile Flower Extract | 0.12.0 |
| White Willow Bark Extract | 0.12.5 |
| Red Clover Flower Extract | 0.12.5 |
| Resveratrol | 0.252.0 |
| Elder Flower Extract | 0.12.0 |
| Aldenine (0.1% GHK Tripeptide1) | 0.253.0 |
| (Hydrolyzed Soy &Wheat Protein) | |

Preservatives/Fragrance

| | |
|---|---|
| PhenoxyEthanol | 0.51.5 |
| DMDM Hydantoin | 0.10.5 |
| Fragrance | N/A |

The composition of the above topical example can be used to form a first mixture which can then be applied alone or with a second mixture administered orally. This second mixture can comprise L-Glutathione and/or Vitamin C. L-Glutathione can be present in the range of 100-600 Mg, while Vitamin C can be provided in a range of 100-800 mg. In at least one embodiment the second mixture can comprise 300 mg of L-Glutathione and 400 mg of Vitamin C.

### Example 3

### Another Example of the First Mixture is Provided for a Topical Composition Such as in a Cream in a Range of Wt % Composition

### Ingredients: Wt %

Water Phase

| | |
|---|---|
| Deionized Water | 35-60 |
| Allantoin | 0.252.0 |
| Na2 EDTA | 0.20.4 |
| Xanthan Gum | 0.251.5 |
| Sodium Lactate | 0.52.5 |
| Citric Acid | 0.254.0 |
| Glycerine 99.5% | 0.255.0 |
| Tetrahydrocurcuminoid | 0.12.0 |
| Tetrahydropiperine | 0.12.0 |
| Glabridin | 0.252.0 |

Oil Phase

| | |
|---|---|
| Sorbitan Oleate | 1.06.0 |
| Glyceryl Stearate/PEG100 Stearate | 0.58.0 |
| Cetearyl Glucoside | 0.57.0 |
| Stearyl Alcohol | 0.258.0 |
| Oleic Acid | 0.255 |
| Glyceryl MonoStearate | 0.258.0 |
| Red Raspberry Seed oil | 0.258.0 |
| BHT | 0.012.5 |
| Dimethicone 200/350 | 0.55.0 |
| Abyssinian Oil | 0.257.0 |
| CoEnzyme Q10 | 0.255.0 |
| Dimthylmethoxy Chromanyl Palmitate | 0.23.5 |
| Undecylenoyl Phenyl Alanine | 0.252.5 |
| Tetrahexyldecyl Ascorbate | 0.253.5 |
| Finsolv TN | 0.253.5 |
| Octocrylene | 2.08 |
| Benzophenone3 | 0.253.0 |

Emulsion Phase

| | |
|---|---|
| Niacinamide | 0.55.0 |

Water

| | |
|---|---|
| Kojic Acid Dipalmitate | 0.253.0 |

Additional Components

| | |
|---|---|
| Aloe Vera Powder | 0.14.0 |
| Datepalm kernel Extract | 0.254.0 |
| Mulberry Bark Extract | 0.14.0 |
| Edelweiss Extract | 0.13.0 |
| Alpha Lipoic Acid | 0.254.0 |
| DMAE | 0.254.5 |
| Sunspheres SPF Booster | |

Low Temp Extracts

| | |
|---|---|
| DLAlpha Tocopherol | 0.254.0 |
| Calendula Flower Extract | 0.12.0 |
| Chamomile Flower Extract | 0.12.5 |
| White Willow Bark Extract | 0.13.5 |
| Red Clover Flower Extract | 0.13.5 |
| Resveratrol | 0.252.5 |
| Elder Flower Extract | 0.13.0 |
| Aldenine (0.1% GHK Tripeptide1) | 0.254.0 |
| (Hydrolyzed Soy &Wheat Protein) | |

Preservatives/Fragrance

| | |
|---|---|
| PhenoxyEthanol | 0.51.5 |
| DMDM Hydantoin | 0.10.5 |
| Fragrance | |

The composition of the above topical example can be used to form a first mixture which can then be applied alone or with a second mixture administered orally. This second mixture can comprise L-Glutathione and/or Vitamin C. L-Glutathione can be present in the range of 100-600 Mg, while Vitamin C can be provided in a range of 100-800 mg. In at least one embodiment the second mixture can comprise 300 mg of L-Glutathione and 400 mg of Vitamin C.

### Example 4

### Another Example of a Topically Applied Mixture Such as for a Cream is Provided in a Range of Wt % Composition

Ingredients: Wt %

Water Phase

| | |
|---|---|
| Deionized Water | 30-70 |
| Allantoin | 0.253.0 |
| Na2 EDTA | 0.20.4 |
| Xanthan Gum | 0.252.0 |
| Sodium Lactate | 0.253.0 |
| Citric Acid | 0.255.0 |
| Glycerine 99.5% | 0.256.0 |
| Tetrahydrocurcuminoid | 0.13.0 |
| Tetrahydropiperine | 0.13.0 |
| Glabridin | 0.253 |

Oil Phase

| | |
|---|---|
| Sorbitan Oleate | 0.57.0 |
| Glyceryl Stearate/PEG100 Stearate | 0.510.0 |
| Cetearyl Glucoside | 0.510 |
| Stearyl Alcohol | 0.2510.0 |
| Oleic Acid | 0.258.0 |
| Glyceryl MonoStearate | 0.2510.0 |
| Red Raspberry Seed oil | 0.259.0 |
| BHT | 0.012.5 |
| Dimethicone 200/350 | 0.258.0 |
| Abyssinian Oil | 0.258.0 |
| CoEnzyme Q10 | 0.257.0 |
| Dimthylmethoxy Chromanyl Palmitate | 0.24.0 |
| Undecylenoyl Phenyl Alanine | 0.253 |
| Tetrahexyldecyl Ascorbate | 0.254.0 |
| Finsolv TN | 0.254.0 |
| Octocrylene | 1.59.0 |
| Benzophenone3 | 0.253.5 |

Emulsion Phase

| | |
|---|---|
| Niacinamide | 0.56.0 |

Water

| | |
|---|---|
| Kojic Acid Dipalmitate | 0.14.0 |

Ingredients: Range 3

Additional Ingredients

| | |
|---|---|
| Aloe Vera Powder | 0.15.0 |
| Date palm kernel Extract | 0.255.0 |
| Mulberry Bark Extract | 0.15.0 |
| Edelweiss Extract | 0.14.0 |
| Alpha Lipoic Acid | 0.25.0 |
| DMAE | 0.255.0 |
| Sunspheres SPF Booster | |

Low Temp Extracts

| | |
|---|---|
| DLAlpha Tocopherol | 0.255.0 |
| Calendula Flower Extract | 0.13.0 |
| Chamomile Flower Extract | 0.13.0 |
| White Willow Bark Extract | 0.15.5 |
| Red Clover Flower Extract | 0.14.5 |
| Resveratrol | 0.253.5 |
| Elder Flower Extract | 0.14. |
| Aldenine (0.1% GHK Tripeptide1) | 0.15.0 |
| (Hydrolyzed Soy &Wheat Protein) | |

Preservatives/Fragrance

| | |
|---|---|
| PhenoxyEthanol | 0.51.5 |
| DMDM Hydantoin | 0.10.5 |
| Fragrance | |

The composition of the above topical example can be used to form a first mixture which can then be applied alone or with a second mixture administered orally. This second mixture can comprise L-Glutathione and/or Vitamin C. L-Glutathione can be present in the range of 100-600 Mg, while Vitamin C can be provided in a range of 100-800 mg. In at least one embodiment the second mixture can comprise 300 mg of L-Glutathione and 400 mg of Vitamin C.

Another composition for the cleanser can be used as well. This composition can includes plurality of different ingredients, however these ingredients are also highlighted by being “preferred” and “non-essential” ingredients. Non-essential ingredients do not have an indicator positioned next to them and are optional. In addition, the associated functions and features of these components are also listed:

| Preferred | Ingredients | Minimum | Maximum | Functions |
|---|---|---|---|---|
| * | Deiodinized Water | To make it | To make it | Solvent |
| | | to 100% | to 100% | |
| * | DMSO | 2 | 40 | Penetration enhancer |
| | Water Cres Extract | 0.5 | 3 | Anti-Oxidant & cancer protection |
| * | Silica Microspheres | 2 | 10 | Exfoliant |
| * | olivem 400 | 5 | 15 | Surfactant |
| * | Olivem 300 | 2 | 15 | Surfactant |
| | Blam Mint Extract | 0.5 | 3 | Anti-bacterial & anti inflammatory |
| * | Lavender Extract | 0.5 | 3 | Anti-bacterial & anti-acne |
| | Clove Extract | 0.1 | 2 | Anti-Oxidant, anti-bacterial |
| | Yarrow Extract | 0.5 | 3 | anti-bacterial & anti inflammatory |
| * | Propylene Glycol | 5 | 35 | Solvent & skin conditioner |
| * | Glycerine | 1 | 5 | Humectant |
| | Dow Corning ES 5227 | 2 | 15 | Skin conditioner |
| * | Aloevera Powder X 100 | 0.05 | 0.2 | Soothes the skin |
| | Saicylic Acid | 0.5 | 2 | Exfoliant |
| | Citric Acid | 0.5 | 2 | pH balance & peeling effect |
| | BHT | 0.1 | 0.75 | to balance Silicone oils & stabilizer |
| * | Lauryl Glucoside | 0.5 | 8 | Surfactant |
| | Decyl Glucoside | 0.5 | 8 | Surfactant |
| * | Stearic Acid | 1 | 4 | emulsifier & skin conditioner |
| | DMAE | 0.25 | 2 | Firming up of skin |
| | PEG 100 Stearate | 0.5 | 5 | emulsifier & skin conditioner |
| * | FCO . . . oil | 8 | 30 | Skin softener & conditioner |
| * | Octyl Palmitate | 1 | 5 | emulsifier & skin conditioner |
| * | C 12-15 Alkyl Benzoate | 0.5 | 3 | smoothes the consistency |
| * | Cetyl Alcohol | 0.5 | 4 | emulsifier & skin conditioner |
| * | Glyceryl Stearate | 0.5 | 5 | emulsifier & skin conditioner |
| * | Retinal Palmitate | 0.5 | 4 | anti-wrinkle & skin rejuvenator |
| * | Cetyl Ricionolate | 0.5 | 2 | moisturizer, conditioner & stabilizer |
| * | Pantathenol | 0.5 | 2 | Skin conditioner & skin vitamin |
| * | Allantoin | 1 | 4 | Skin conditioner, softener & anti acne |
| | Carbomer | 0.5 | 5 | Thickener |
| * | Tocopherol | 0.5 | 4 | Vitamin E for skin |
| * | Xanthan Gum | 0.25 | 5 | stabilizer |
| * | Methyl Paraban | 0.1 | 0.5 | preservative |
| * | Propyl Paraban | 0.1 | 0.5 | preservative |
| * | Di sodium EDTA | 0.2 | 0.4 | Chelating agent |
| | Fragrance | As Desired | | |

The ingredients of the cleanser can also be used with an additional toner. The preferred ingredients for this toner are listed with a “*” positioned adjacent to this ingredient. An example of a toner that can be used with the above cleanser is listed below in wt %, and which also lists its functions for each component:

Anti Aging Toner

| Preferred | Ingredient | Minimum % | Maximum % | Functions |
|---|---|---|---|---|
| * | Deionized Water | To make it | To make it | |
| | | to 100% | to 100% | |
| * | Witch Hazel | 5 | 20 | Anti-Inflammatory & astringent |
| * | SD 40 Alcohol | 10 | 40 | Anti-bacterial, astringent |
| * | Allantoin | 1 | 3 | Anti-inflammatory, anti-acne |
| | Willow Bar Extract | 0.5 | 5 | Anti-inflammatory, astringent, exfoliant |
| * | Aloe | 1 | 3 | Anti-acne, anti-oxidant, soothes the skin |
| * | Caprylyl Glycol | 1 | 3 | moisturizer & humectant |
| * | Citric Acid | 0.5 | 2 | pH adjuster, anti-wrinkle |
| | Phenoxy Ethanol | 1 | 2 | preservative |
| | Polysorbate 20 | 0.5 | 2 | Dispersing agent |
| | Sorbic acid | 0.5 | 2 | Preservative |
| * | Di sodium EDTA | 0.1 | 0.4 | Chelating agent |
| * | DMSO | 1 | 10 | Skin penetration enhancer |
| | Fragrance | As Desired | | |

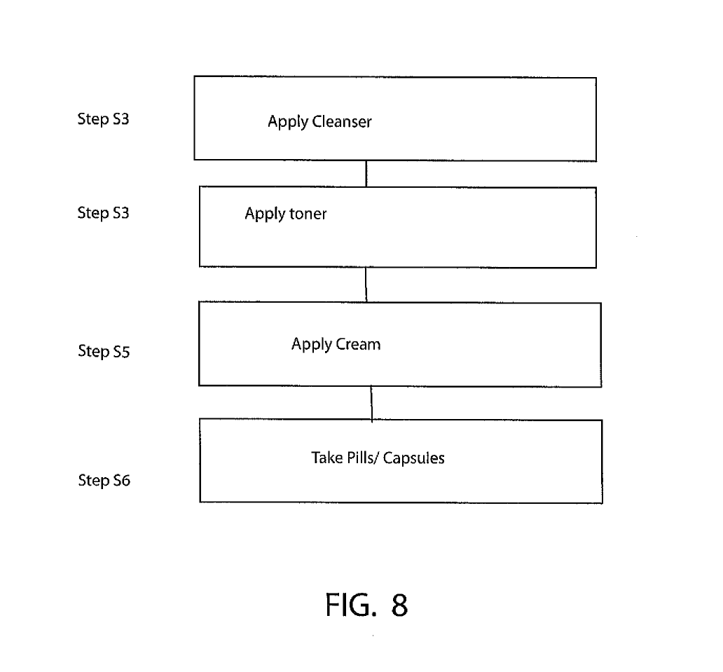

As shown in FIG. 1, the topical application can be used together with other non-topical applications as well. The topical applications can include any one of an astringent, cleanser, toner, or cleanser. For example FIG. 8 shows an additional process can also be used to apply the components listed above as well. FIG. 8 starts with step S3 by applying a cleanser to a person's skin such as a persons' face. Next, in step S4 a person can apply an astringent to clean the face after the application of a cleanser. Next in step S5 the person can apply a toner to firm the skin and prepare it for the anti-aging application listed above. This application can be in the form of a cream, an oil, or any other type of fluid or solid type of application as well in step S6. Next, in step S7, the user can ingest or take the oral composition as listed above. Therefore, this comprehensive process thereby creates a system for a person to reduce the effects or to inhibit the effects of aging by first preparing the person's skin and then applying a topical anti-aging application to the prepared skin while still attacking the effects of aging internally on the person's body as well.

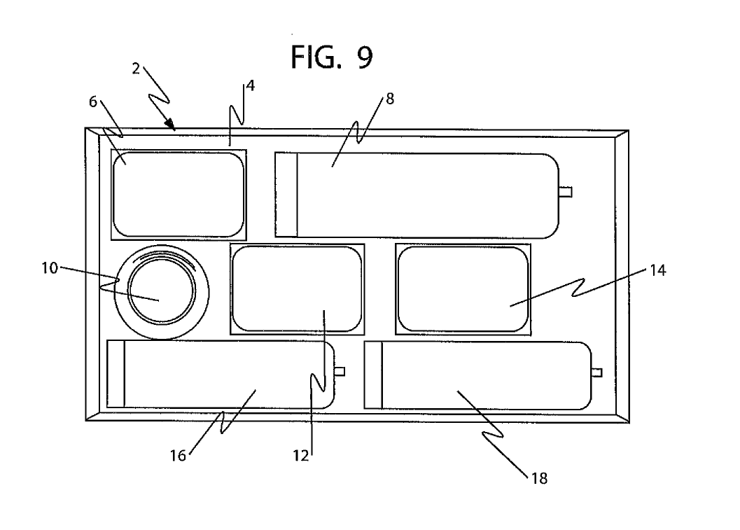

In addition to aid the user in this application the above components that are to be applied can be presented as part of a kit. For example, as shown in FIG. 9, there is a kit 2 which is comprises a kit container 4 which includes a plurality of components used to carry out the process listed above. For example, there is a first set of applicators or application pads 6 which can either be infused with a cleanser or toner. Next, there is shown an anti-aging topical application container 8 which can be in the form of a tube or container which is used to allow a user to apply this material to the skin. Disposed in this kit container 4 is also a bottle 10 which is configured to house caplets or pills which are configured to allow the user to administer the oral composition such as vitamin c and L-glutathione. There are also two different sets of application pads 12 and 14 which can be either infused with material or be used to apply different materials such as a cleanser, a toner or a cream to apply the anti-aging material as well. Containers 16 and 18 can be used to hold any one of the cleanser material the toner material or the cream as well.

The cleanser can be used to further promote the helpful effects of the solution provided above, and some of the materials disclosed in the cleanser can be used in conjunction with the solution indicated above and as disclosed as being in tubes 8. The solution of the cleanser can include the following:

| Ingredients | Minimum wt % | Maximum wt % |
|---|---|---|
| Water | 20 | 60 |
| DMSO | 2 | 40 |
| Water Cress Extract | 0.5 | 3 |
| Soap Bark Extract | 0.5 | 10 |
| Olivem 300 | 5 | 15 |
| Blam Mint Extract | 0.5 | 3 |
| Lavender Extract | 0.5 | 3 |
| Clove Extract | 0.1 | 2 |
| Yarrow Extract | 0.5 | 3 |
| FCO oil | 2 | 10 |
| Glycerine | 1 | 5 |
| Dimethacone | 0.5 | 4 |
| Aloe vera Powder X 100 | 0.05 | 0.2 |
| Saicylic Acid | 0.5 | 2 |
| Citric Acid | 0.5 | 2 |
| BHT | 0.1 | 0.75 |
| Stearic Acid | 1 | 4 |
| DMAE | 0.25 | 2 |
| PEG 100 Stearate | 0.5 | 5 |
| Octyl Palmitate | 1 | 5 |
| C 12-15 Alkyl Benzoate | 0.5 | 3 |
| Cetyl Alcohol | 0.5 | 4 |
| Glyceryl Stearate | 0.5 | 5 |
| Retinal Palmitate | 0.5 | 4 |
| Cetyl Ricionolate | 0.5 | 2 |
| Pantathenol | 0.5 | 2 |
| Allantoin | 1 | 4 |
| Carbomer | 0.5 | 5 |
| Tocopherol | 0.5 | 4 |
| Xanthan Gum | 0.25 | 5 |
| Methyl Paraban | 0.1 | 0.5 |
| Propyl Paraban | 0.1 | 0.5 |
| Di sodium EDTA | 0.2 | 0.4 |
| Fragrance | As Desired | |

In addition, the toner as disclosed above can include the following ingredients with the following wt % composition:

| Ingredient | Minimum % | Maximum % |
|---|---|---|
| Water | 30 | 80 |
| Witch Hazel | 5 | 20 |
| SD 40 Alcohol | 10 | 40 |
| Allantoin | 1 | 3 |
| Willow Bar Extract | 0.5 | 5 |
| Aloe | 1 | 3 |
| Caprylyl Glycol | 1 | 3 |
| Citric Acid | 0.5 | 2 |
| Phenoxy Ethanol | 1 | 2 |
| Polysorbate 20 | 0.5 | 2 |
| Sorbic acid | 0.5 | 2 |
| Di sodium EDTA | 0.1 | 0.4 |
| DMSO | 1 | 10 |
| Fragrance | As Desired | |

While the compositions provided above are included as examples, these are only examples and different compositions can be mixed and matched to achieve the desired effect disclosed above.

Therefore, this kit provides an inclusive kit for the application of an anti-aging material to assist a user in applying this material to their body. In this way, by following a specific process using a specific formula, the user can turn back the hands of time and look to reverse the appearance of aging by selectively removing unwanted pigmentation, wrinkles, puffiness of the skin, while reviving the tone and presence of the person's skin in earlier years.

Accordingly, while at least one embodiment of the present invention has been shown and described, it is to be understood that many changes and modifications may be made thereunto without departing from the spirit and scope of the invention as defined in the appended claims.

What is claimed is:

1. An anti-aging composition comprising:
a mixture comprising:
a plurality of active ingredients comprising:
a plurality of pigmentation reducing elements;
a plurality of anti-oxidants;
a plurality of anti-wrinkling agents;
a plurality of anti-inflammatory agents;
at least one additional agent comprising at least one of an emollient, a proactant and a conditioning agent.

2. The composition as in claim 1, wherein said plurality of pigmentation reducing elements comprises at least one of: Tetrahydrocumin; Undecylenoyl Phenyl Alanine, Dimethylmethoxy Chromanyl Palmitate, Niacinamide, Kojic Acid Dipalmitate, Tetrahexydecyl Ascorbate, Glabridin, Mulberry bark Extract, TransResveratrol.

3. The composition as in claim 1, wherein said plurality of anti oxidents comprises: at least one of at least one of CoQ10;Alpha Lipoic Acid; Aloe vera Extract; Green Tea extract; DLAlpha Tocopherol; Edelweiss Extract; or Tetrahydrocurcuminoid.

4. The composition as in claim 1, wherein said plurality of anti-wrinkling agents comprises at least one of: Tripeptidel, Hydrolyzed soy and Wheat Proteins; Date Palm Kernel Extract; Dimethylaminoethanol (DMAE); or Resveratrol/TransResveratrol.

5. The composition as in claim 1, wherein said plurality of anti inflammatory agents comprises at least one of: Calendula; Chamomile; or white willow bark extract.

6. The composition as in claim 1, wherein said at least one additional agent comprises: an emollient, a proactant, and a conditioning agent including at least one of Glycerine; allantoin; Sodium Lactate; Abyssinian Oil; Red Raspberry oil; water, Sorbitan Oleate, Glyceryl Stearate, PEG100 Stearate, Cetearyl Glucoside, Stearyl Alcohol, Oliec Acid, & Glyceryl Monostearate.

7. The composition as in claim 1, wherein the mixture is contained in a topical emulsion.

8. The composition as in claim 7, further comprising a second mixture comprising at least one of L-Glutathione, and Vitamin C.

9. The composition as in claim 7, wherein said second mixture is contained in a capsule and is configured for oral ingestion.

10. The composition as in claim 9, wherein said second mixture comprises between 100 mg 600 mg of LGlutathione.

11. The composition as in claim 9, wherein said second mixture comprises between 100 mg 800 mg of Vitamin C.

12. The composition of claim 9, wherein said second mixture comprises 300 mg of LGlutathione and 400 mg of Vitamin C.

13. The composition of claim 9, wherein said first mixture comprises 400 Mg of LGlutathione and 500 mg of Vitamin C.

14. The composition of claim 1, wherein said first mixture comprises a topically applied composition comprising the following ingredients:
Deionized Water 3070 wt %;
Allantoin 0.253.0 wt %;
Na2 EDTA 0.20.4 wt %;
Xanthan Gum 0.252.0 wt %;
Sodium Lactate 0.253.0 wt %;
Citric Acid 0.255.0 wt %;
Glycerine 99.5% 0.256.0 wt %;
Tetrahydrocurcuminoid 0.13.0 wt %;
Tetrahydropiperine 0.13.0 wt %;
Glabridin 0.253 wt %;
Sorbitan Oleate 0.57.0 wt %;
Glyceryl Stearate/PEG100 Stearate 0.510.0 wt %;
Cetearyl Glucoside 0.510 wt %;
Stearyl Alcohol 0.2510.0 wt %;
Oleic Acid 0.258.0 wt %;
Glyceryl MonoStearate 0.2510.0 wt %;
Red Raspberry Seed oil 0.259.0 wt %;
BHT 0.012.5 wt %;
Dimethicone 200/350 0.258.0 wt %;
Abyssinian Oil 0.258.0 wt %;
CoEnzyme Q10 0.257.0 wt %;
Dimthylmethoxy Chromanyl Palmitate 0.24.0 wt %;
Undecylenoyl Phenyl Alanine 0.253 wt %;
Tetrahexyldecyl Ascorbate 0.254.0 wt %;
Finsolv TN 0.254.0 wt %;
Octocrylene 1.59.0 wt %;
Benzophenone3 0.253.5 wt %;
Emulsion Phase wt %;
Niacinamide 0.56.0 wt %;
Kojic Acid Dipalmitate 0.14.0 wt %;
Ingredients: Range 3 wt %;
Aloe Vera Powder 0.15.0 wt %;
Datepalm kernel Extract 0.255.0 wt %;
Mulberry Bark Extract 0.15.0 wt %;
Edelweiss Extract 0.14.0 wt %;
Alpha Lipoic Acid 0.25.0 wt %;
DMAE 0.255,0 wt %;
Sunspheres SPF Booster wt %;
Low Temp Extracts wt %;
DLAlpha Tocopherol 0.255.0 wt %;
Calendula Flower Extract 0.13.0 wt %;
Chamomile Flower Extract 0.13.0 wt %;
White Willow Bark Extract 0.15.5 wt %;
Red Clover Flower Extract 0.14.5 wt %;
Resveratrol 0.253.5 wt %;
Elder Flower Extract 0.14. wt %;
Aldenine (0.1% GHK Tripeptide l) 0.15.0 wt %;
PhenoxyEthanol 0.51.5 wt %; and
DMDM Hydantoin 0.10.5 wt %.

15. The composition of claim 1, wherein said first mixture comprises a topically applied composition comprising the following ingredients:
Deionized Water 3560 wt %;
Allantoin 0.252.0 wt %;
Na2 EDTA 0.20.4 wt %;
Xanthan Gum 0.251.5 wt %;
Sodium Lactate 0.52.5 wt %;
Citric Acid 0.254.0 wt %;
Glycerine 99.5% 0.255.0 wt %;
Tetrahydrocurcuminoid 0.12.0 wt %;
Tetrahydropiperine 0.12.0 wt %;
Glabridin 0.252.0 wt %;
Sorbitan Oleate 1.06.0 wt %;
Glyceryl Stearate/PEG100 Stearate 0.58.0 wt %;
Cetearyl Glucoside 0.57.0 wt %;
Stearyl Alcohol 0.258.0 wt %;
Oleic Acid 0.255 wt %;
Glyceryl MonoStearate 0.258.0 wt %;
Red Raspberry Seed oil 0.258.0 wt %;
BHT 0.012.5 wt %;
Dimethicone 200/350 0.55.0 wt %;
Abyssinian Oil 0.257.0 wt %;
CoEnzyme Q10 0.255.0 wt %;
Dimthylmethoxy Chromanyl Palmitate 0.23.5 wt %;
Undecylenoyl Phenyl Alanine 0.252.5 wt %;
Tetrahexyldecyl Ascorbate 0.253.5 wt %;
Finsolv TN 0.253.5 wt %;
Octocrylene 2.08 wt %;
Benzophenone3 0.253.0 wt %;
Niacinamide 0.55.0 wt %;
Kojic Acid Dipalmitate 0.253.0 wt %;
Aloe Vera Powder 0.14.0 wt %;
Datepalm kernel Extract 0.254.0 wt %;
Mulberry Bark Extract 0.14.0 wt %;
Edelweiss Extract 0.13.0 wt %;
Alpha Lipoic Acid 0.254.0 wt %;
DMAE 0.254.5 wt %;
DLAlpha Tocopherol 0.254.0 wt %;
Calendula Flower Extract 0.12.0 wt %;
Chamomile Flower Extract 0.12.5 wt %;
White Willow Bark Extract 0.13.5 wt %;
Red Clover Flower Extract 0.13.5 wt %;
Resveratrol 0.252.5 wt %;
Elder Flower Extract 0.13.0 wt %;
Aldenine (0.1% GHK Tripeptidel) 0.254.0 wt %;
PhenoxyEthanol 0.51.5 wt %; and
DMDM Hydantoin 0.10.5 wt %.

16. The composition of claim 1, wherein said first mixture comprises a topically applied composition comprising the following ingredients:
Deionized Water 3070 wt %;
Allantoin 0.253.0 wt %;
Na2 EDTA 0.20.4 wt %;
Xanthan Gum 0.252.0 wt %;
Sodium Lactate 0.253.0 wt %;
Citric Acid 0.255.0 wt %;
Glycerine 99.5% 0.256.0 wt %;
Tetrahydrocurcuminoid 0.13.0 wt %;
Tetrahydropiperine 0.13.0 wt %;
Glabridin 0.253 wt %;
Sorbitan Oleate 0.57.0 wt %;
Glyceryl Stearate/PEG 100 Stearate 0.510.0 wt %;
Cetearyl Glucoside 0.510 wt %;
Stearyl Alcohol 0.2510.0 wt %;
Oleic Acid 0.258.0 wt %;
Glyceryl Mono Stearate 0.2510.0 wt %;
Red Raspberry Seed oil 0.259.0 wt %;
BHT 0.012.5 wt %;
Dimethicone 200/350 0.258.0 wt %;
Abyssinian Oil 0.258.0 wt %;
CoEnzyme Q10 0.257.0 wt %;
Dimthylmethoxy Chromanyl Palmitate 0.24.0 wt %;
Undecylenoyl Phenyl Alanine 0.253 wt %;
Tetrahexyldecyl Ascorbate 0.254.0 wt %;
Finsolv TN 0.254.0 wt %;
Octocrylene 1.59.0 wt %;
Benzophenone3 0.253.5 wt %;
Emulsion Phase wt %;
Niacinamide 0.56.0 wt %;
Kojic Acid Dipalmitate 0.14.0 wt %;
Aloe Vera Powder 0.15.0 wt %;
Datepalm kernel Extract 0.255.0 wt %;
Mulberry Bark Extract 0.15.0 wt %;
Edelweiss Extract 0.14.0 wt %;
Alpha Lipoic Acid 0.25.0 wt %;
DMAE 0.255,0 wt %;
Sunspheres SPF Booster wt %;
DLAlpha Tocopherol 0.255.0 wt %;
Calendula Flower Extract 0.13.0 wt %;
Chamomile Flower Extract 0.13.0 wt %;
White Willow Bark Extract 0.15.5 wt %;
Red Clover Flower Extract 0.14.5 wt %;
Resveratrol 0.253.5 wt %;
Elder Flower Extract 0.14. wt %;
Aldenine (0.1% GHK Tripeptidel) 0.15.0 wt %;
(Hydrolyzed Soy &Wheat Protein) wt %;
PhenoxyEthanol 0.51.5 wt %; and
DMDM Hydantoin 0.10.5 wt %.

17. The composition of claim 1, wherein said first mixture comprises a topically applied composition comprising the following ingredients:
Deionized Water 44 wt %;
Allantoin 0.5 wt %;
Na2 EDTA 0.2 wt %;
Xanthan Gum 0.5 wt %;
Sodium Lactate 1.5 wt %;
Citric Acid 1 wt %;
Glycerine 99.5% 2.5 wt %;
Tetrahydrocurcuminoid 0.25 wt %;
Tetrahydropiperine 0.25 wt %;
Glabridin 0.5 wt %;
Sorbitan Oleate 3.5 wt %;
Glyceryl Stearate/PEG100 Stearate 3.5 wt %;
Cetearyl Glucoside 2 wt %;
Stearyl Alcohol 0.5 wt %;
Oleic Acid 0.5 wt %;
Glyceryl MonoStearate 2 wt %;
Red Raspberry Seed oil 2 wt %;
BHT 0.05 wt %;
Dimethicone 200/350 1.5 wt %;
Abyssinian Oil 1.5 wt %;
CoEnzyme Q10 2.5 wt %;
Dimthylmethoxy Chromanyl Palmitate 1 wt %;
Undecylenoyl Phenyl Alanine 1 wt %;
Tetrahexyldecyl Ascorbate 2 wt %;
Finsolv TN 1.7 wt %;
Octocrylene 5 wt %;
Benzophenone3 1.5 wt %;
Niacinamide 1 wt %;
Water 6 wt %;
Kojic Acid Dipalmitate 0.5 wt %;
Aloe Vera Powder 0.5 wt %;
Datepalm kernel Extract 1.5 wt %;
Mulberry Bark Extract 0.2 wt %;
Edelweiss Extract 0.2 wt %;
Alpha Lipoic Acid 1 wt %;
DMAE 1 wt %;
DLAlpha Tocopherol 1 wt %;
Calendula Flower Extract 0.2 wt %;
Chamomile Flower Extract 0.2 wt %;
White Willow Bark Extract 0.2 wt %;
Red Clover Flower Extract 0.2 wt %;
Resveratrol 1 wt %;
Elder Flower Extract 0.2 wt %;
Aldenine (0.1% GHK Tripeptide l) 1 wt %;
PhenoxyEthanol 1 wt %; and
DMDM Hydantoin 0.15 wt %.

18. A method for reducing the effects of aging on a person's skin comprising:
Applying a topical composition at least twice daily comprising:
Deionized Water 3070 wt %;
Allantoin 0.253.0 wt %;
Na2 EDTA 0.20.4 wt %;
Xanthan Gum 0.252.0 wt %;
Sodium Lactate 0.253.0 wt %;
Citric Acid 0.255.0 wt %;
Glycerine 99.5% 0.256.0 wt %;
Tetrahydrocurcuminoid 0.13.0 wt %;
Tetrahydropiperine 0.13.0 wt %;
Glabridin 0.253 wt %;
Sorbitan Oleate 0.57.0 wt %;
Glyceryl Stearate/PEG 100 Stearate 0.510.0 wt %;
Cetearyl Glucoside 0.510 wt %;
Stearyl Alcohol 0.2510.0 wt %;
Oleic Acid 0.258.0 wt %;
Glyceryl MonoStearate 0.2510.0 wt %;
Red Raspberry Seed oil 0.259.0 wt %;
BHT 0.012.5 wt %;
Dimethicone 200/350 0.258.0 wt %;
Abyssinian Oil 0.258.0 wt %;
CoEnzyme Q10 0.257.0 wt %;
Dimthylmethoxy Chromanyl Palmitate 0.24.0 wt %;
Undecylenoyl Phenyl Alanine 0.253 wt %;
Tetrahexyldecyl Ascorbate 0.254.0 wt %;
Finsolv TN 0.254.0 wt %;
Octocrylene 1.59.0 wt %;
Benzophenone3 0.253.5 wt %;
Emulsion Phase wt %;
Niacinamide 0.56.0 wt %;
Kojic Acid Dipalmitate 0.14.0 wt %;
Ingredients: Range 3 wt %;
Aloe Vera Powder 0.15.0 wt %;
Datepalm kernel Extract 0.255.0 wt %;
Mulberry Bark Extract 0.15.0 wt %;
Edelweiss Extract 0.14.0 wt %;
Alpha Lipoic Acid 0.25.0 wt %;
DMAE 0.255,0 wt %;
Sunspheres SPF Booster wt %;
Low Temp Extracts wt %;
DLAlpha Tocopherol 0.255.0 wt %;
Calendula Flower Extract 0.13.0 wt %;
Chamomile Flower Extract 0.13.0 wt %;
White Willow Bark Extract 0.15.5 wt %;
Red Clover Flower Extract 0.14.5 wt %;
Resveratrol 0.253.5 wt %;
Elder Flower Extract 0.14. wt %;
Aldenine (0.1% GHK Tripeptidel) 0.15.0 wt %;
PhenoxyEthanol 0.51.5 wt %; and
DMDM Hydantoin 0.10.5 wt %; and
consuming an oral supplement comprising between 100-600 mg of L-Glutathione and 100-800 mg of Vitamin C.

19. The method as in claim 18 wherein said topical composition comprises:
Deionized Water 44 wt %;
Allantoin 0.5 wt %;
Na2 EDTA 0.2 wt %;
Xanthan Gum 0.5 wt %;
Sodium Lactate 1.5 wt %;
Citric Acid 1 wt %;
Glycerine 99.5% 2.5 wt %;
Tetrahydrocurcuminoid 0.25 wt %;
Tetrahydropiperine 0.25 wt %;
Glabridin 0.5 wt %;
Sorbitan Oleate 3.5 wt %;
Glyceryl Stearate/PEG100 Stearate 3.5 wt %;
Cetearyl Glucoside 2 wt %;
Stearyl Alcohol 0.5 wt %;
Oleic Acid 0.5 wt %;
Glyceryl Mono Stearate 2 wt %;
Red Raspberry Seed oil 2 wt %;
BHT 0.05 wt %;
Dimethicone 200/350 1.5 wt %;
Abyssinian Oil 1.5 wt %;
CoEnzyme Q10 2.5 wt %;
Dimthylmethoxy Chromanyl Palmitate 1 wt %;
Undecylenoyl Phenyl Alanine 1 wt %;
Tetrahexyldecyl Ascorbate 2 wt %;
Finsolv TN 1.7 wt %;
Octocrylene 5 wt %;
Benzophenone3 1.5 wt %;
Niacinamide 1 wt %;
Water 6 wt %;
Kojic Acid Dipalmitate 0.5 wt %;
Aloe Vera Powder 0.5 wt %;
Datepalm kernel Extract 1.5 wt %;
Mulberry Bark Extract 0.2 wt %;
Edelweiss Extract 0.2 wt %;
Alpha Lipoic Acid 1 wt %;
DMAE 1 wt %;
DLAlpha Tocopherol 1 wt %;
Calendula Flower Extract 0.2 wt %;
Chamomile Flower Extract 0.2 wt %;
White Willow Bark Extract 0.2 wt %;
Red Clover Flower Extract 0.2 wt %;
Resveratrol 1 wt %;
Elder Flower Extract 0.2 wt %;
Aldenine (0.1% GHK Tripeptidel) 1 wt %;
PhenoxyEthanol 1 wt %; and
DMDM Hydantoin 0.15 wt %; and
said oral composition comprises 300 mg of LGlutathione and 400 mg.
